(19) United States
(12) Patent Application Publication
(10) Pub. No.: US 2010/0298579 A1
(43) Pub. Date: Nov. 25, 2010

(54) Process for preparing synthetic cannabinoids

(75) Inventors: CHRISTIAN STEUP, HOFHEIM (DE); THOMAS HERKENROTH, BRANNENBURG (DE)

Correspondence Address: CANTOR COLBURN LLP, 20 Church Street, 22nd Floor, Hartford, CT, 06103, US

(73) Assignee: THC PHARM GMBH, FRANKFURT (DE)

(21) Appl. No.: 12/770,056

(22) Filed: Apr. 29, 2010

(30) Foreign Application Priority Data
Apr. 29, 2009 (DE) 10 2009 019 322.7

(51) Int. Cl. C07D 311/80; C07C 39/12; C07C 49/00

(52) U.S. Cl. 549/390; 568/729; 568/329

(57) ABSTRACT

The field of the invention is organic synthesis, more particularly a process for preparing cannabinoids. The process described is applicable to all stereoisomers and homologues of cannabinoids.

For this purpose, the present patent application provides a process for preparing the abovementioned compounds in two or three chemical synthesis steps.

### FIELD OF THE INVENTION

The field of the disclosure is organic synthesis, more particularly a process for preparing cannabinoids. The process described is applicable to all stereoisomers and homologues of cannabinoids.

### BACKGROUND

Since the discovery of indigenous receptors, cannabinoids have moved increasingly into the field of interest of pharmaceutical research. Cannabidiol (1a) (the numbers between brackets which follow the compounds specified in all of the text relate to the structural formulae shown below in tables 1 and 2), delta-9-tetrahydrocannabinol (dronabinol) (2a) and nabilone (rac. trans-4b), and the isomers and homologues thereof, have a series of pharmacological properties which make them substances of therapeutic interest.

Cannabidiol is additionally of particular economic significance as a starting substance for the synthesis of dronabinol.

### SUMMARY

The disclosure provides a novel process for preparing the abovementioned compounds with few process steps and with a good yield.

For this purpose, the disclosure provides a process for preparing the abovementioned compounds in two or three chemical synthesis steps.

### DETAILED DESCRIPTION

In a first step (“a”) compounds of the general formula III (e.g. alkylresorcyl esters (6-alkyl-2,4-dihydroxybenzoic esters, 5a)) are condensed with unsaturated hydrocarbons, alcohols, ketones (or derivatives thereof such as enol esters, enol ethers and ketals) in high yields to give the corresponding 3-substituted 6-alkyl-2,4-dihydroxybenzoic esters.

Examples of this kind of reactions have been described, inter alia, by Crombie, L. et al. in J. Chem. Research (S) 114, (M), pp. 1301-1345 (1977), and have been referred to there as acid-catalysed terpenylation.

In a second step (“b”), the intermediates with an ester function obtained in the first step are subjected to a decarboxylating hydrolysis, which forms the corresponding ester-free cannabinoids.

If necessary or desired, in a third step (“c”) an acid-catalysed rearrangement is undertaken. This isomerization may, for example, be the ring closure of the pyran ring in the case of CBD to give dronabinol, but also the rearrangement of a double bond, for example the rearrangement of delta-9- to delta-8-THC, or an acid-catalysed epimerisation such as the rearrangement of cis-9-ketocannabinoids to the corresponding trans compounds.

The acid-catalysed rearrangement c, where necessary, may also precede the hydrolysis step b.

In the context of the disclosure, step (b) in particular should be emphasized, since it is novel and inventive. Thus, the disclosure provides achievement via by a proposed process for preparing a compound of the general formula I, especially Ia, Ib or Ic and diastereoisomers thereof

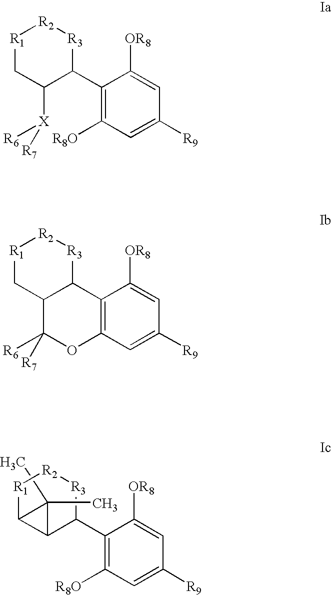

in which decarboxylating hydrolysis of the compound of the general formula IIa, IIb, IIc or IId

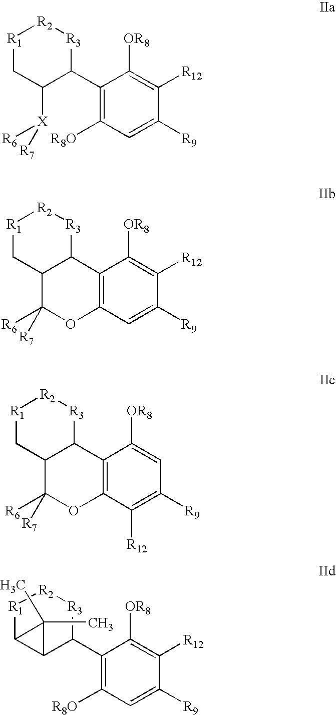

where
R1-R2 or R2-R3 may be a C—C double bond, and
R1 and R3 are each C or CH, and
R2 is either C═O or an R10-C—R11 group,
   where
R10 and R11 are each independently H or a lower C₁-C₄ alkyl group when a double bond is not present between R1 and R3, or, if a double bond is present between R1 and R3, one of the R10 and R11 groups is absent and the other is as defined above;
X is either C when R6 is a ═CH₂ group and R7 is a CH₃ group, or
X is a CR4 group where R4 is H or a lower alkyl group, CH or a C—O—R5 group, and R5 is H, a C₁-C₁₆ alkyl group or a protecting group;
R6 and R7 are each a CH₃ group or at least one of the R6 and R7 groups is a CH2=group and the other is a CH₃ group,
R8 is a C₁-C₁₆ alkyl group, H or a protecting group,
R9 is a C1-C16 or O—C₁-C₁₆ group, where C₁-C₁₆ is a straight or branched alkyl chain which has one or more double or triple bonds at any position or may have substituents such as deuterium or halogen atoms, phenyl, substituted phenyl, cycloalkyl, nitrile, alkoxy or a keto group,
R12 is a CO₂—R13 group, and
R13 is H, a C₁-C₁₆ alkyl group or a protecting group,
affords cannabinoids.

In order to convert the ester intermediates of the condensation step “a” to the end products, it is necessary to hydrolyse and to decarboxylate the ester group of the condensation products of the first stage (step “b”).

An acidic hydrolysis of the ester group is not an option in the cases in which the desired cannabinoid formed tends to undesired isomerization under acidic conditions, as, for example, in the case of CBD or delta-9-THC, and the stereoisomers thereof and homologues thereof.

In the compounds of the 16 type, for example cannabidiolcarboxylic esters, acidic treatment forms a large amount of undesired by-products such as delta-8-tetrahydrocannabinol and isotetrahydrocannabinols (Israel Journal of Chemistry, Vol. 6, 1968, 679-690).

This is also true of the preparation of delta-9-tetrahydrocannabinol from the esters of delta-9-tetrahydrocannabinolic acid A or B (formula images 17a and 18a).

This is of course also true of the homologues of the cannabinoids mentioned.

It is therefore the case that the direct synthesis of delta-9-tetrahydrocannabinol from delta-9-tetrahydrocannabinolic esters by hydrolysis also cannot be performed in an acidic medium owing to the tendency of the double bond in the “8” position to migrate.

One alternative to the acidic hydrolysis of the ester group is alkaline hydrolysis (step “b”).

In the case of the ketocannabinoids too, such as nabilone, the stereoisomers thereof and the homologues thereof, it is possible to apply the processes for hydrolysis and decarboxylation described here under “b”, which afford superior yields of the desired products.

For the ester precursors of the ketocannabinoids such as nabilone, an acidic hydrolysis of the precursors, for example by boiling with aqueous mineral acid in a suitable solvent such as acetic acid, is possible in principle and leads in the case of the cis compounds of the 23 and 24-A and -B types (formula images 23, 24A, 24B) to the epimerisation to the corresponding trans-cannabinoids.

However, epimerisation of the ester precursors with acids forms a lower level of by-products than in the case of treatment of the ester-free end products.

The alkaline hydrolysis of the ester group with subsequent decarboxylation allows preparation of cannabinoids of the verbenyl olivetolate type (formula image 21a) or compounds of the 35 type (formula image 35) without isomerization.

This optimally utilizes the advantage of higher regioselectivity achieved in the synthesis of cannabinoids with alkylresorcyl esters compared to a synthesis with alkylresorcinols, and the increased stability of the intermediates with an ester group.

In the case of compounds which tend to isomerization (ring closure and/or migration of the double bond) under acidic conditions, such as CBD-carboxylic esters ([formula image 16], R1=n-C5H11) and CBD ([formula image 1], R1=n-C5H11) or the esters of the tetrahydrocannabinolic acids A and B ([17a] and [18a]), a correspondingly optimized process for hydrolysis and decarboxylation of the ester intermediates is instrumental in making the synthesis route described at the outset economically viable.

However, owing to the acidity of the phenolic hydroxyl groups in the reaction of the ester intermediates with alkalis, phenoxide anions form, which are relatively resistant to further attack by hydroxide anions.

The process described in the literature, boiling esters of the [16] type (Petrzilka et al., Helv. Chim. Acata 52 (1969), 1102-1134) or [18] type (Herlt et al., U.S. Pat. No. 5,342,971) with alkalis in methanol, in our experience afforded substantially unchanged starting material and only low yields of the desired product even after very long reaction times of several days. It is therefore unusable for an economic synthesis of active pharmaceutical ingredients and precursors thereof.

On the industrial scale, tank occupation time is a crucial factor which decides the economic viability of a process.

The presence of unreacted starting material also necessitates complex purification steps such as chromatography in the cases in which the product is otherwise preparable in pure form by less expensive processes such as distillation and crystallization.

There was therefore a search for an economic process which leads to a full conversion of the starting material within a few hours and affords the desired hydrolysed and decarboxylated products with good yields.

We have now found that the ester precursors of the cannabinoids can be hydrolysed and decarboxylated in outstanding yields and virtually without formation of by-products to give the corresponding end products when one of the following processes (summarized here under reaction step “b”) is employed:
1. A pressure process which allows the employment of elevated temperatures with simultaneous use of low-boiling solvents such as lower alcohols and the mixture thereof with water.
   Suitable solvents for the pressure process are lower alcohols having one up to five carbon atoms, ammonia, and the mixtures thereof with one another and with water. It is likewise possible to employ pure water with a phase transfer catalyst or emulsifier.
2. An ambient pressure process which allows a reaction regime in an “open” system.
   Suitable solvents for the ambient pressure process are water-miscible solvents with a boiling point above 100° C. at standard pressure, for example dimethylformamide, dimethyl sulphoxide, sulpholane, furfurol, di- and tetrahydrofurfurol, 2-methoxytetrahydrofuran, hexamethylenephosphoramide, acetamide and other amides having up to 12 carbon atoms, tetramethylurea, ethylene glycol, and the mono- and bisethers thereof, ethanolamine, ethylenediamine, propylene glycol and ethers thereof having up to 20 carbon atoms, glycerol and glyceryl ethers having up to 30 carbon atoms, 1,2-butanediol, 1,3-butanediol, 1,4-butanediol, diethylene glycol and ethers thereof, triethylene glycol and ethers thereof, polyethylene glycol and polyvinylpyrrolidone, and the mixtures thereof with one another and with water.
   For both processes:
   Bases suitable for alkaline hydrolysis are the hydroxides of alkali metals and alkaline earth metals and of quaternary ammonium salts, the carbonates, hydrogencarbonates, carboxylates having one up to 30 carbon atoms, phenoxides, phosphates, phosphites, sulphides, hydrogensulphides, mercaptides of one up to 30 carbon atoms, sulphites, hydrogensulphites, cyanides of alkali metals and quaternary ammonium salts having four to 48 carbon atoms.
   Likewise suitable are ammonia and organic amines (including pyridine bases) having one up to 36 carbon atoms, and the salts thereof, and also borates such as sodium tetraborate (borax) and other basic salts.
3. The use of catalysts (polyvalent cations, and finely divided transition metals and salts thereof) which accelerate the decarboxylation of the acids formed as intermediates after the hydrolysis (cannabinoid carboxylic acids) and hence remove these acids from the reaction equilibrium.

Suitable catalysts are finely divided transition metals and the salts of transition metals, for example stainless steel powder or silver powder.

The advantage of the pressure process lies in the easy removability of low-boiling solvents from the reaction products by distillation, which facilitates the recycling of the solvent and makes the process more environmentally friendly.

The advantage of the ambient pressure process lies in the lower apparatus complexity which arises from the reaction regime in an open system compared to a pressure vessel.

In order to prepare the intermediates IIa, IIb, IIc and IId, in a first step a, alkylresorcyl esters (6-alkyl-2,4-dihydroxybenzoic esters) (formula image 5) are condensed with unsaturated hydrocarbons, alcohols, ketones (or derivatives thereof such as enol esters, enol ethers and ketals) in high yields to give the corresponding 3-substituted 6-alkyl-2,4-dihydroxybenzoic esters.

In a second step b, the intermediates with an ester function obtained in the first step are subjected to a decarboxylating hydrolysis, which forms the corresponding ester-free cannabinoids.

If necessary, in a third step c an acid-catalysed rearrangement is undertaken. This isomerization may, for example, be the ring closure of the pyran ring in the case of CBD to give dronabinol, but also the rearrangement of a double bond, for example the conversion of delta-9- to delta-8-THC or an acid-catalysed epimerisation such as the rearrangement of cis-9-ketocannabinoids to the corresponding trans compounds.

Where necessary, the acid-catalysed rearrangement c may also precede the hydrolysis step b.

It is thus also possible to subject the ester precursors which are finished in terms of the structure of the hydrocarbon skeleton to the decarboxylating hydrolysis.

R¹ is a straight or branched alkyl chain or alkoxy chain of one up to 16 carbon (C) atoms, which may have double bonds, triple bonds or further substituents such as deuterium atoms, phenyl groups, substituted phenyl groups, cycloalkyl groups, nitrile groups, alkoxy groups and keto groups at any position.

R² is a carboxyl protecting function (definition analogous to Herlt U.S. Pat. No. 5,342,971 p. 4) of one up to 16 carbon atoms, typically an alkyl function or a substituted alkyl function such as benzyl (phenylmethyl), diphenyl methyl or 2-substituted alkyl radicals of one to 16 carbon atoms, such as (i) lower alkoxy, e.g. 2-methoxyethyl, 2-ethoxyethyl, (ii) lower alkylthio, for example 2-methylthioethyl and 2-ethylthioethyl, (iii) halogen such as 2,2,2-trichloroethyl, 2-bromoethyl and 2-chloroethyl, (iv) alkyl groups substituted by one or two phenyl groups (substituted or unsubstituted); and aroyl groups such as phenacyl.

Table I shows examples of the compounds which are obtained by the process:

TABLE I

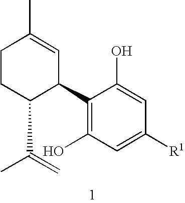

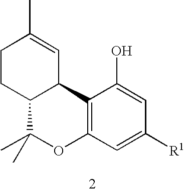

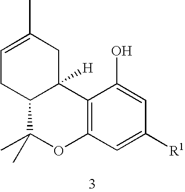

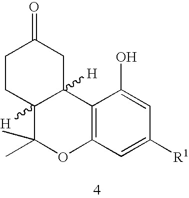

where, for example:
a: R¹=n-C₅H₁₁ (corresponds to 1)
(−)-CBD corresponds to 1a:
2-((1R,6R)-3-Methyl-6-(prop-1-en-2-yl)cyclohex-2-enyl)-5-pentylbenzene-1,3-diol(−)-delta-9-THC corresponds to 2a:
(6aR,10aR)-6,6,9-Trimethyl-3-pentyl-6a,7,8,10a-tetrahydro-6H-benzo[c]chromen-1-ol-delta-8-THC corresponds to 3a:
(6aR,10aR)-6,6,9-Trimethyl-3-pentyl-6a,7,10,10a-tetrahydro-6H-benzo[c]chromen-1-ol
b: R1=1,1-dimethylheptyl-
Nabilone=racemic trans corresponds to 4b:
racemic trans-1-hydroxy-6,6-dimethyl-3-(2-methyloctan-2-yl)-7,8,10,10a-tetrahydro-6H-benzo[c]chromen-9(6aH)-one

Use of suitable reactants from Table II allows cannabinoid carboxylic esters to be formed in the manner detailed above.

Table II containing compounds (5) to (15) gives an overview of possible unsaturated hydrocarbons, alcohols and ketones (or derivatives thereof, such as enol esters, enol ethers and ketals) usable for condensation (step a).

A keto function may be protected as the enol ether, enol ester or ketal, where R³ and R⁴ may each be straight-chain, branched or cyclic organic groups having up to 16 carbon atoms or organosilicon radicals having up to 16 carbon atoms.

R³ and R⁴ may also be straight-chain or branched hydrocarbon radicals which are bridged to one another and have up to 16 carbon atoms, for example —(CH₂)ₙ—, —CH₂(CCH₃)₂CH₂—.

The R⁵ and R⁶ groups may be [formula type (6) to (15)] hydrogen (H) or an alcoholic protecting function such as a straight-chain, branched or cyclic alkyl, acyl or organosilicon radical having up to 16 carbon atoms.

Particular emphasis is due to the fact that, in the case of the compounds detailed, ethers and esters (R⁵ and R⁶=straight-chain, branched or cyclic alkyl, acyl, organosilyl each having up to 16 carbon atoms), under the conditions of the acidic terpenylation (reaction step “a”), can also react like the corresponding free alcohols (R⁵ and R⁶═H).

When terpenes with optically active substitution are used in the menthane structural moiety on C-4, it is possible to prepare optically active cannabinoids as end products (cf. also T. Petrzilka et al.: Helv. Chinn. Acta Vol. 52 (1969) 1102-1134).

The same applies to the bicycloheptanes and bicycloheptenes such as verbenol (8a; R⁵═H), apoverbenone (14) or compounds of the (15) type, when compounds clearly defined in terms of the configuration at the C1 and C5 bridgehead atoms are used.

These condense with (5) with retention of the absolute configuration at C4 and C5 and it is thus possible to prepare optically active ester intermediates and cannabinoids.

TABLE II
(reactants for synthesis step a):

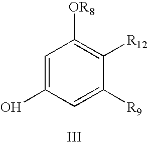

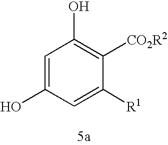

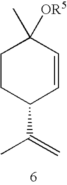

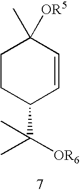

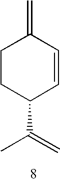

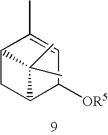

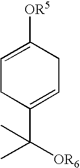

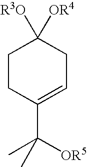

10 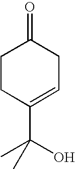
11 “4-(2-hydroxypropan-2-yl)cyclohex-3-enone”

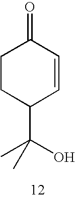

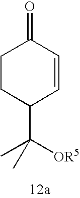

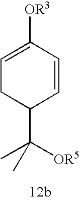

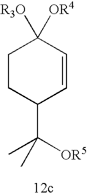

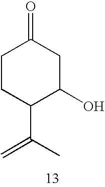

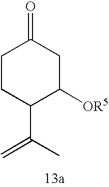

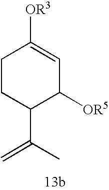

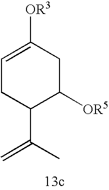

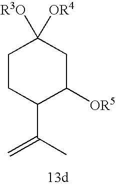

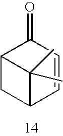

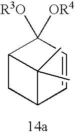

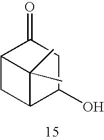

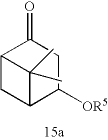

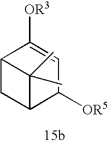

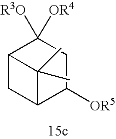

Examples for the formulae of Table II:
6-Alkyl-2,4-dihydroxybenzoic ester (alkylresorcyl ester) (5a):
5a corresponds to the general formula III, when R8 is H and R1 and R2 correspond to the substituents R9 and R12 of the formula III (R8 is a C₁-C₁₆ alkyl group, H or a protecting group, R9 is a C1-016 or O—C₁-C₁₆ group, where C₁-0₁₆ is a straight or branched alkyl chain which has one or more double or triple bonds at any position or may have substituents such as deuterium or halogen atoms, phenyl, substituted phenyl, cycloalkyl, nitrile, alkoxy or a keto group, R12 is a CO₂—R13 group, and R13 is H, a C₁-C₁₆ alkyl group or a protecting group).
cis- and trans-p-Mentha-2,8-dien-1-ol (6a, R⁵═H)
p-Mentha-2-ene-1,8-diol (7a, R⁵═R⁶═H)
Menthatriene (8)
(−)-trans-Verbenol (9a, R⁵═H)

Structures of the 10 type obtainable from 4-methoxyacetophenone by Grignard and subsequent Birch reduction, for example 10a:
2-(4-Methoxycyclohexa-1,4-dienyl)propan-2-ol (10a; R⁵═CH₃, R⁶═H)

Structures of the 11 type, for example: 2-(4,4-dimethoxycyclohex-1-enyl)propan-2-ol (11a, R³═R⁴═CH₃; R⁵═H); these can be interpreted as a masked “4-(2-hydroxypropan-2-yl)cyclohex-3-enone”.

4-(2-Hydroxypropan-2-yl)cyclohex-2-enone (12) and the masked forms thereof 12a to 12c, 3-hydroxy-4-(prop-1-en-2-yl)cyclohexanone (13) and the masked forms thereof 13a to 13d.

6,6-Dimethylbicyclo[3.1.1]hept-3-en-2-one (apoverbenone) (14) and the masked forms thereof, for example 6,6-dimethyl-2,2-diacetoxy-3-norpinene (13a, R³═R⁴═COCH₃)

4-Hydroxy-6,6-dimethylbicyclo[3.1.1]heptan-2-one (15) and the masked forms thereof, for example 6,6-dimethyl-2,4-diacetoxy-2-norpinene (15b, R³═R⁵═COCH₃)

It is possible to use the compounds 6 to 9 to form cannabinoids of the 1 to 3 types, whereas the compounds 10 to 15 can be used to form 9-ketocannabinoids of the 4 type, for example nabilone.

The intermediates formed by reaction a have an ester function CO₂R², and are referred to hereinafter as “ester intermediates”.

In a subsequent reaction step, the ester intermediates are converted to the corresponding ester-free cannabinoids which bear a hydrogen function in place of the ester function.

In some cases, an acid-catalysed rearrangement (reaction step c) is also necessary to synthesize the desired end product.

This rearrangement may be an isomerization or, as a special case thereof, an epimerisation and may either precede or follow reaction step b.

The acid-catalysed rearrangement c and the acidic terpenylation can in some cases also advantageously be performed as a “one-pot process”, such that the rearranged intermediates can be subjected to the decarboxylating hydrolysis b.

When, for example, compounds of the 1 type are condensed by method a with (+)-cis- or trans-menthadienol 6a (R⁵═H) or esters thereof, ester intermediates of the 16 type form in outstanding yields, for example the cannabidiol acid methyl ester 16a (R¹=n-C₅H₁₁; R²═CH₃).

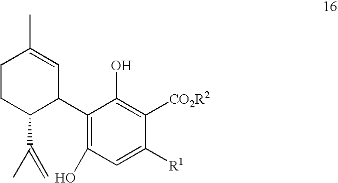

It is possible to prepare cannabidiol (la; R¹=n-C₅H₁₁) therefrom by decarboxylating hydrolysis and, in a subsequent acid-catalysed isomerization step c, (−)-delta-9-tetrahydrocannabinol (2a; R¹=n-C₅H₁₁).

It is possible to prepare delta-8-tetrahydrocannabinol (3a; R¹=n-C₅H₁₁) from the latter by a prolonged contact time of the acidic catalyst.

However, it is also possible to perform the isomerization step before the decarboxylating hydrolysis c.

For instance, the two positional isomers delta-9-tetrahydrocannabinolic ester-A 17 and delta-9-tetrahydrocannabinolic ester-B 18 (in each case R¹=n-C₅H₁₁; R²═CH₃) are obtained from the condensation product (16a) of methyl olivetolate (5a R¹=n-C₅H₁₁; R²═CH₃) with menthadienol 6a (R⁵═H) or esters thereof.

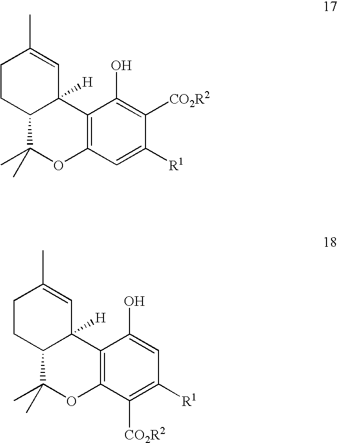

Prolonged contact time of the catalyst leads here too to the rearrangement of the double bond to the 8 position:

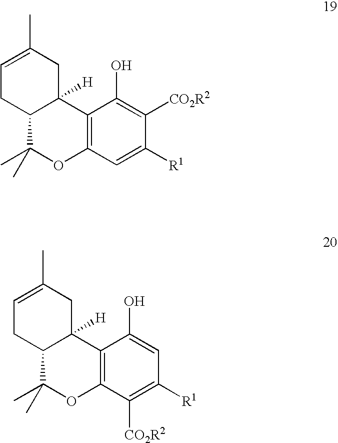

For instance, delta-9-tetrahydrocannabinolic ester-A (17) forms delta-8-tetrahydrocannabinolic ester-A (19) and delta-9-tetrahydrocannabinolic ester-B (18) forms delta-8-tetrahydrocannabinolic ester-B (20) (in each case R¹=n-C₅H₁₁; R²═CH₃).

It is possible to obtain compounds of the (2) type by method b from the compounds (17) and (18).

It is possible to obtain compounds of the (3) type by method b from the compounds (19) and (20).

The same reactions can be carried out with (7) and (8) as with (6).

(−)-trans-Verbenol (9a; R⁵═H) and esters thereof were condensable with methyl olivetolate (5a; R¹=n-C₅H₁₁; R²═CH₃) to give “para”-verbenyl olivetolate (21a; R¹=n-C₅H₁₁; R²═CH₃) (reaction “a”) from which, under acid catalysis (reaction “c”), delta-8-tetrahydrocannabinolic acid-A methyl ester (19a) and delta-8-tetrahydrocannabinolic acid-B methyl ester (20a) were prepared (Crombie refers to these as delta-^{1,6}-tetrahydrocannabinolic acid-A and -B methyl ester).

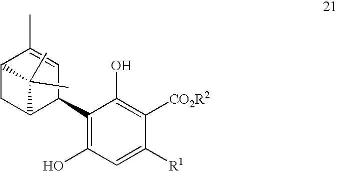

The hydrolysis and decarboxylation (process b) of verbenyl olivetolate described here allows preparation of verbenyl olivetol (22a; R¹=n-C₅H₁₁), from which it is possible to prepare, by means of acid-catalysed isomerization (process “c”), delta-8- or delta-9-THC.

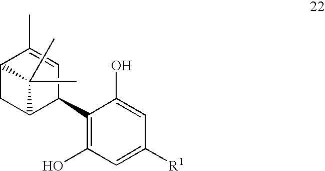

From methyl olivetolate (R¹=n-C₅H₁₁, R²═CH₃) and geraniol, it is possible to prepare, by acidic condensation, cannabigerolic acid methyl ester (methyl cannabigerolate) and, from this, by the process described under b, cannabigerol (CBG). It is thus possible to prepare the cannabichromenoic acid methyl esters A and B from citral. Both give cannabichromene by process b.

Compare, for example, the preparation of cannabigerol via cannabigerolic ester and of cannabichromene via cannabichromenoic esters-A and -B on pages 19 and 20.

The synthesis route specified here is particularly suitable for preparing the lower homologues of CBD and tetrahydrocannabinol, for example cannabidivarol CBDV, R¹=n-C₃H₇ and delta-9-tetrahydrocannabivarol THCV, R¹=n-C₃H₇, since the ester group in the condensation step “a” suppresses the formation of positional isomers (R¹=n-C₃H₇).

In the case of the ketocannabinoids too, for example nabilone, and the stereoisomers thereof and homologues thereof, it is possible to advantageously employ the processes described here for condensation “a” of alcohols, ketones (or derivatives thereof, such as enol ethers, enol esters and ketals), carboxylic acids and esters with compounds of the III type, which afford, on completion of hydrolysis and decarboxylation (process “b”), superior yields of the desired products.

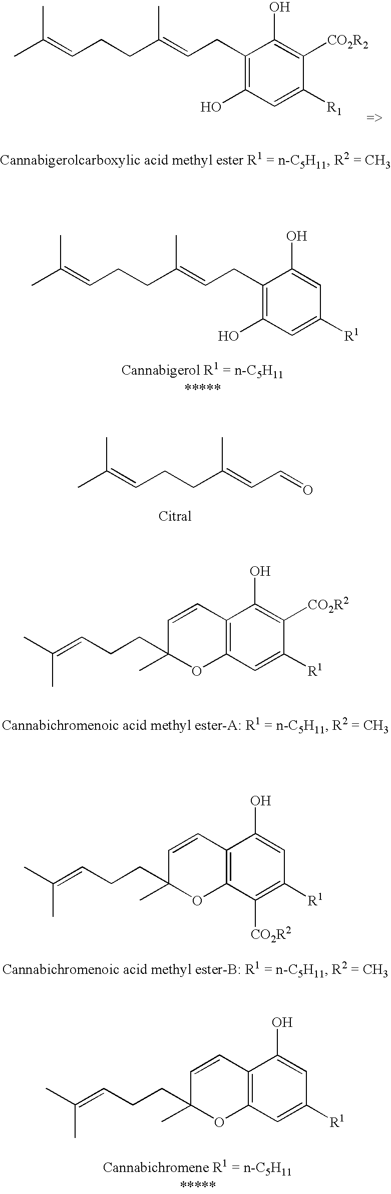

The following condense with 5:

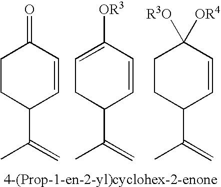

When, for example, compounds of the (5) type are reacted with 2-(4-methoxy-1,4-cyclohexadienyl)-2-propanol (10a; R⁵═R⁶═CH₃) according to process “a”, this forms the ester precursors of nabilone and homologues thereof in cis-form: (23-A), [23] as a racemic mixture of the positional isomers ester A (23-A and 24-A) and B (23-B and 24-B)

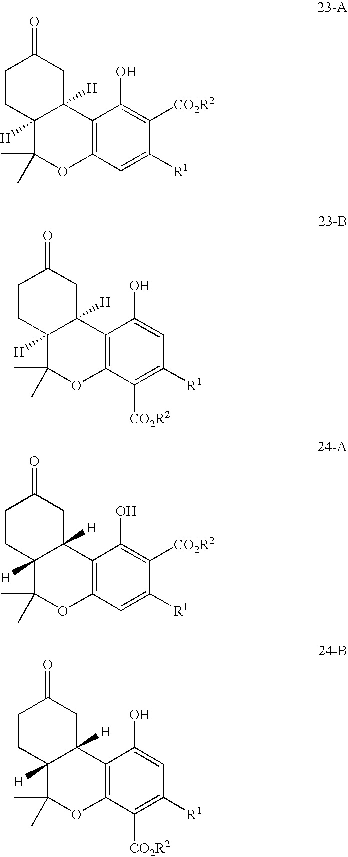

According to the catalyst and solvent used, the condensation step also forms acetals (25-A) and (25-B) or (26-A) and (26-B).

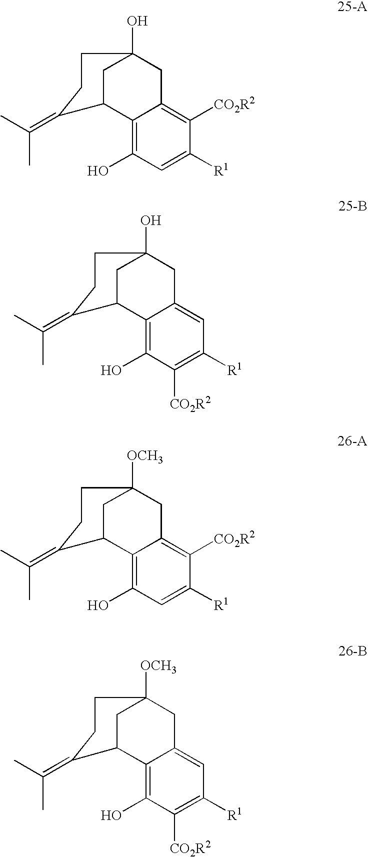

23-A and 24-A form, by acid-catalysed epimerisation, the racemate of the trans-esters 27-A and 28-A (27-A and 28-A as a racemic mixture):

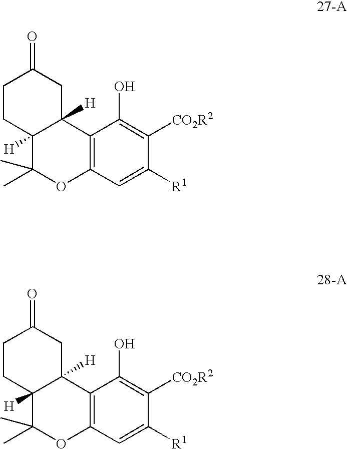

23-B and 24-B form, by acid-catalysed epimerisation, the racemate of the trans-esters 27-B and 28-B (27-B and 28-B as a racemic mixture):

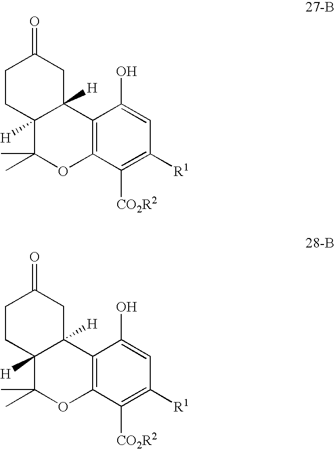

The acetals 25 and 26 form, by acid-catalysed rearrangement, the mixture of the cis-esters 23 and 24, which can be rearranged further under acidic conditions to the trans-esters 27 and 28.

Alkaline decarboxylating hydrolysis (step b) forms, from the trans compounds 27 and 28, racemic compounds of the trans-(4) type, for example nabilone (R¹=1,1-dimethylheptyl)

The cis compounds 23 and 24 can also, like the acetals 25 and 26 too, first be subjected to the decarboxylating hydrolysis, and the rearrangement, analogously to Archer et al.: (J. Org. Chem. Vol. 42 pp. 1177-2284), can be conducted on the corresponding ester-free cis compounds.

23-A and -B thus form 29:

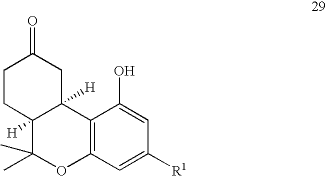

24-A and -B thus form 30:

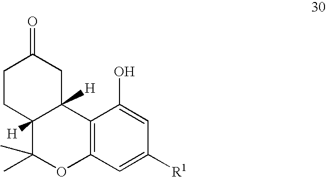

The acetals 25 form compounds of the 31 type:

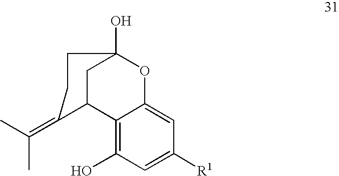

The acetals of the 26 type form compounds of the 32 type:

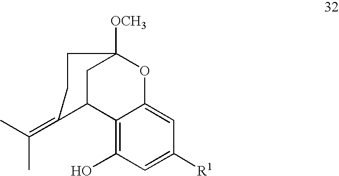

31 and 32 can, as described in Archer et al., be rearranged either directly or via the cis compounds' acid catalysis to the compounds of the trans-4 type.

In this case, the keto function may be protected as the enol ether, enol ester or ketal, where R³ and R⁴ may each be straight-chain, branched or cyclic organic groups having up to 16 carbon atoms or organosilicon radicals having up to 16 carbon atoms.

R³ and R⁴ may also be straight-chain or branched hydrocarbon radicals which are bridged to one another and have up to 16 carbon atoms, for example —(CH₂)ₙ—, —CH₂(CCH₃)₂CH₂—.

The R⁵ and R⁶ groups may (type 10 to 15) be hydrogen (H) or an alcoholic protecting function such as a straight-chain, branched or cyclic alkyl, acyl or organosilicon radical having up to 16 carbon atoms.

Particular emphasis is due to the fact that, in the case of the compounds detailed, ethers and esters (R⁵ and R⁶=straight-chain, branched or cyclic alkyl, acyl, organosilyl having in each case up to 16 carbon atoms) can also react under the conditions of the acid terpenylation (reaction step “a”) like the corresponding free alcohols (R⁵ and R⁶═H).

Optically Active Enantiomers of Nabilone and Homologues Thereof:

When 12 or 13 with optically active substitution in the 4 position or compounds 14 or 15 with unambiguous steric definition at C5, for example (1S,5S)-6,6-dimethylbicyclo[3.1.1]hept-3-en-2-one=[(+)-apoverbenone]=(1S,5S)-14:

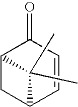

(1S,5S)-14
(1S,5S)-6,6-dimethylbicyclo[3.1.1]hept-3-ene-2,2-diyldiacetate=(1 S,5S)-14a (R³═R4=CO—CH₃)[=(+)-6,6-dimethyl-2,2-diacetoxy-3-norpinene]:

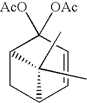

(1S,5S)-14a (R³═R4=CO—CH₃)
   or
(1S,5R)-6,6-dimethylbicyclo[3.1.1]hept-2-ene-2,4-diyldiacetate[=(−)-6,6-dimethyl-2,4-diacetoxy-2-norpinene]=(1S,5S)-15b, (R³═R⁵═CO—CH₃)

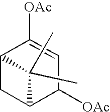

(1S,5S)-15b, (R³═R⁵═CO—CH₃)
   are condensed with compounds of the 5 type, it is possible to form optically active 9-ketocannabinoids while retaining the absolute configuration of the C4 in the menthane skeleton or at the C5 of the bicyclo[3.3.1]heptene.

(+)-Apoverbenone=(1S,5S)-14 condenses with compounds of the 5 type directly to give the optically active trans-esters 28-A and 28-B, which, after decarboxylating hydrolysis b, give the corresponding ester-free compounds 33:

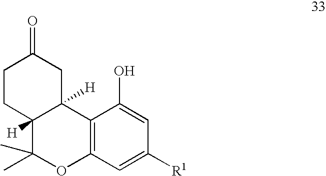

(1S,5S)-14a and (1S,5S)-15b condense with 5 to give compounds of the 34 type:

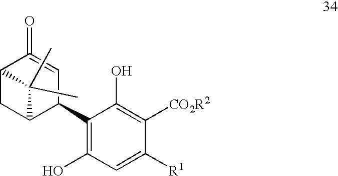

34 can be rearranged under acid catalysis (e.g. with SnCl₄) to compounds of the 28-A and -B type, from which 33 form after decarboxylating hydrolysis.

Here too, the decarboxylating hydrolysis (step b) may take place before the acid-catalysed rearrangement.

In the latter case, 34 is first used to prepare 35, which is then rearranged under acid catalysis to 33.

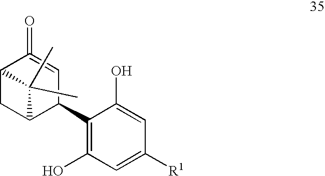

Quite generally, it is possible for all products presented here for which an acid-catalysed rearrangement is part of the synthesis method to perform this rearrangement before or after the decarboxylating hydrolysis “b”.

Acids suitable for the condensation step (step “a”) are both Brønsted acids and Lewis acids:

Examples of Suitable Brønsted Acids:

Perchloric acid, hydrohalic acids (HF, HCl, HBr, HI), sulphuric acid, hydrogensulphates, phosphoric acid and the acidic salts thereof, pyro- and polyphosphoric acids, organic carboxylic and sulphonic acids having one up to 30 carbon atoms and one or more acidic groups, and acidic groups bonded to polymeric supports, for example acidic ion exchangers and mixtures of the acids mentioned. Specific examples include formic acid, oxalic acid, trifluoroacetic acid, p-toluenesulphonic acid.

Examples of Suitable Lewis Acids:

The cations of alkaline earth metals and earth metals, and also transition metals; the halogen compounds and other trivalent compounds of elements of the third main group, such as boron trifluoride and other boron-halogen compounds and complexes thereof, aluminium halides such as anhydrous aluminium chloride;
salts and halogen compounds of transition metals such as titanium tetrachloride, zinc chloride, zinc trifluoromethanesulphonate;
halogen compounds of elements of the fourth and fifth and sixth main groups, for example tin tetrachloride, phosphorus trichloride, phosphorus pentachloride, phosphorus oxychloride, antimony pentafluoride, thionyl chloride, sulphuryl chloride, alone or in a mixture with other Lewis or Brønsted acids. Positive sites bound to polymeric frameworks, such as montmorillonite,

Further suitable reagents for performing the condensation are the acetals of N,N-dimethylformamide, for example N,N-dimethylformamide dineopentyl acetal and other water-releasing reagents, for example those as used for the formation of amides and peptides, for example “T3P” (propanephosphonic anhydride).

These reagents can be added as such to the reaction mixture or be applied to a support material, for example aluminium oxide.

Suitable solvents for performing the condensation step are water-immiscible or water-miscible solvents, for example hydrocarbons having up to 30 carbon atoms, halogenated hydrocarbons having up to 20 carbon atoms, for example dichloromethane or chloroform, ethers, for example 2-methoxytetrahydrofuran, alcohols, carboxylic acids having up to 16 carbon atoms, amides having up to 20 carbon atoms, esters having up to 60 carbon atoms, carbon dioxide, sulphur dioxide, water, water with a phase transfer catalyst, the acidic catalysts themselves, and mixtures of the solvents mentioned with one another.

The acids and solvents mentioned are also used for the isomerization and epimerization reactions mentioned (reaction step “c”); in that case, generally somewhat more energetic conditions are selected, for example higher temperatures.

The Disclosure is Now Illustrated in Detail by the Examples

The condensation process (“a”) and the two methods for decarboxylating hydrolysis (“b”) are explained hereinafter using the preparation of cannabidiol (CBD) from p-mentha-2,8-dien-1-ol and methyl olivetolate (methyl 6-n-pentyl-2,4-dihydroxybenzoate) as an example.

One example of a rearrangement (isomerization, reaction step “c”) is the synthesis of dronabinol from CBD.

Step 1: Condensation of p-mentha-2,8-dienol with methyl olivetolate (method “a”):

This step is identical whether followed by hydrolysis and decarboxylation by the pressure process or at ambient pressure, or whether a subsequent isomerization “c” takes place before or after the hydrolysis “b”.

A 10 litre three-neck flask with stirrer, reflux condenser, internal thermometer and dropping funnel is initially charged with:
300 g (1.259 mol) of methyl olivetolate
196.4 g (1.290 mol) of p-mentha-2,8-dien-1-ol
2.5 litres of dichloromethane (preferably unstabilized, freshly distilled material)
optionally, a water-binding agent, for example 100 g of anhydrous sodium sulphate or 120 g of magnesium sulphate, can be added.

The mixture is stirred until a homogeneous solution has formed.

The flask is immersed into an external ice-salt cooling bath and stirring is continued until an internal temperature of minus 15° C. has been attained.

A solution of 59.5 g (0.419 mol) of boron trifluoride-diethyl etherate in 500 ml of unstabilized, dry dichloromethane is introduced into the dropping funnel.

The boron trifluoride etherate solution is added dropwise with vigorous stirring and external cooling to the reaction mixture within approx. one hour, in the course of which an internal temperature of approx. minus 15° C. is maintained.

The reaction solution becomes yellowish and turbid.

Once the total amount of boron trifluoride etherate has been added, the mixture is stirred at minus 15° C. for another approx. 15 min.

The flask is removed from the ice bath.

Subsequently, a solution of 180 g (1.8 mol) of potassium hydrogencarbonate in 2 l of deionized water is allowed to run in with vigorous stirring within approx. 30 min, in the course of which evolution of carbon dioxide occurs towards the end.

Stirring is continued for two more hours, then the mixture is transferred to a separating funnel and the aqueous phase (pH approx. 8) is removed and discarded.

The organic phase is washed with two portions each of 1 l of deionized water.

The organic phase is removed and concentrated on a rotary evaporator. Finally, the bath temperature is raised to 90° C. and the pressure is reduced to 3 mbar in order to remove residual solvent.

Yield: 466 g (99%) of crude methyl cannabidiolate (CBDAMe), which contains approx. 10-20% unchanged starting material (methyl olivetolate).

Purification of the Crude Methyl Cannabidiolate (CBDAMe):

466 g of crude methyl cannabidiolate are dissolved with gentle heating (40° C.) in 2 l of a suitable water-immiscible solvent, for example petroleum ether (pe) or methyl tert-butyl ether (MTBE).

The solution is extracted with two portions each of 0.8 l of 0.5 N sodium hydroxide.

The aqueous phases are combined and can be acidified to recover unreacted methyl olivetolate.

The organic phase is washed with two portions each of 0.5 l of deionized water, in each of which 20 g of sodium sulphate may be dissolved in order to improve the phase separation.

Remove the organic phase and concentrate on a rotary evaporator; finally, the bath temperature is raised to 90° C. and the pressure is reduced to 3 mbar in order to remove residual solvent.

Yield: 376 g (80% of theory) of CBDAMe with approx. 80% purity.

Step 2: Hydrolysis and Decarboxylation of the Cannabinoid Carboxylic Esters (Methods “b”)

For this step, the pressure process is first described using the example of methyl cannabidiolcarboxylate: (analogous to TH 338)

A 2 l stainless steel autoclave with a magnetic stirrer and internal thermometer is initially charged with:
74.5 g (0.20 mol) of methyl cannabidiolate
120 ml of deionized water
25.0 g (0.18 mol) of potassium carbonate
180 ml of methanol

The autoclave is purged with argon, sealed and heated on a hot plate with a magnetic stirrer.

Once an internal temperature of 140-150° C. has been attained, the mixture is stirred at this temperature for 4 to 5 hours.

Thereafter, it is allowed to cool to <40° C. and decompressed.

The autoclave contents are transferred with 250 ml of methanol into a round-bottom flask and neutralized by cautious (CO₂ evolution—foaming!) addition of a solution of 23.2 g (0.36 eq.) of citric acid in 150 ml of deionized water.

The emulsion which forms is concentrated on a rotary evaporator (recovery of aqueous methanol), and the residue consisting of CBD, potassium salts of citric acid and residual water is dissolved between 200 ml of deionized water and 300 ml of petroleum ether (or another water-immiscible solvent) by rotating in a water bath at 40°.

Separate phases in a separating funnel, discard aqueous phase and wash the organic phase with 2×100 ml of 3% sodium sulphate solution.

Concentrating the organic phase by rotary evaporation leaves 62.3 g (99% of theory) of crude CBD.

The ambient pressure process for hydrolysis and decarboxylation of the ester intermediates is described hereinafter with reference to CBDAMe (according to TH 502):

The apparatus consists of a 10 l three-neck flask in a heating mantle with stirrer, internal thermometer and a Claisen attachment with 30 cm Vigreux column and gas inlet tube. A distillation attachment with a top thermometer and descending distillation column is mounted on the Vigreux column, which has a graduated flask as a receiver.

The following are introduced successively into the 10 l flask:
540.1 g (1.45 mol) of CBDAMe
2.0 l of monoethylene glycol
a solution of 67.4 g (1.08 mol) of approx. 90% potassium hydroxide flakes in
340 ml of deionized water
0.5 g of stainless steel powder

The apparatus is purged with approx. 5 l of argon/min for 5 min, then heating is commenced while stirring, and the argon stream is reduced to approx. 0.1 l/min.

At an internal temperature of approx. 128° C. the flask contents begin to boil and, soon thereafter, a methanol-rich mixture begins to distil via the top of the column.

Heating is continued cautiously while stirring and introducing inert gas, such that distillate distils over slowly and continuously.

After boiling for approx. 3 h, approx. 140 ml of distillate have formed; the bottom temperature has risen to 140° C. and the top temperature to 100° C.

Allow to cool while introducing inert gas; at the same time, exchange the Vigreux column for a dropping funnel with pressure equalization.

At internal temperature 85° C., add 3 l of deionized water and then, within approx. half an hour, add a solution of 76.5 g (0.40 mol) of citric acid in 1 l of deionized water dropwise (foaming towards the end of the dropwise addition as a result of CO2 evolution!).

Then the inert gas stream is stopped and 1.5 l of petroleum ether (or another water-immiscible solvent) is added dropwise at an internal temperature of <40° C. within half an hour, in the course of which further carbon dioxide escapes.

Stir at high speed for at least 1 h.

Transfer the flask contents to a separating funnel, remove the lower phase and extract with 1 l of petroleum ether (or another water-immiscible solvent).

Combine the organic phases and wash five times with 0.60 l of deionized water, the emulsion-like intermediate phase being breakable by addition of approx. 0.10 l of 10% sodium sulphate solution.

Remove the organic phase, concentrate by rotary evaporation, draw off residual solvent under reduced pressure at a bath temperature of 90° C.

Yield: 455.1 g (100% of theory) of crude CBD

Both for the pressure process and for the ambient pressure process, the same bases can be used for hydrolysis.

Shortening of the Reaction Time by Catalysts:

Both for the ambient pressure process and the pressure process, the reaction times can be shortened by adding a suitable catalyst, for example 0.1% by weight (based on CBDAMe) of stainless steel powder or 0.01% by weight of silver powder. This catalyst accelerates the decarboxylation of the carboxylic acid formed as an intermediate.

The crude product is purified further by one or more of the following processes:
1. distillation, 2. silica gel filtration (chromatography), 3. crystallization and recrystallization.

These processes can be employed individually or in any combination in order to obtain pure CBD.

1. Distillation

The vacuum distillation of CBD can be effected either from a liquid phase flask or from a thin-film apparatus. Appropriately, distillation is effected at pressures below 1 mbar, preferably <0.3 mbar. The cooling liquid of the condenser should be sufficiently warm (>50° C.) to ensure a sufficient downflow rate of the condensed CBD.

Fractional Distillation of CBD from a Liquid-Phase Flask:

Apparatus:

1 l round-bottom flask with heating mantle, stirrer, bottom thermometer and attached distillation system with top thermometer, exchangeable receivers for collecting fractions.

Thermostatic water bath with circulation pump as cooling liquid for the distillation system.

Vacuum pump with manometer and upstream cold trap charged with liquid nitrogen.

Procedure:

The distillation flask is charged with 242 g of crude CBD.

The crude CBD is preheated to approx. 60° C. and the stirrer is started.

Vacuum is then applied cautiously and the bottom temperature is raised slowly.

At a cooling liquid temperature of <30° C., 5 g of first runnings, which consists principally of terpenes, distil at a top temperature of 50-60° C. and a pressure between 3 mbar and 0.8 mbar.

A second fraction (16.4 g) with 68% CBD distils at a top temperature of 120-132° C. and a pressure of 0.70 to 0.14 mbar. Cooling water 60° C.

The third fraction (178 g) consists of 90% CBD and distils at top temperature 133-155° C. and a pressure of 0.10 to 0.15 mbar. Cooling water 70° C.

In the liquid phase flask, 42 g of residue remain with less than 5% CBD.

Short-Path Thin-Film Distillation of CBD:

The dropping funnel of a short-path distillation apparatus like KD 1 from UIC is charged in portions with 1971.4 g of preheated (approx. 60° C.) CBD.

The heating jacket of the apparatus is kept at 180° C.

The cold trap for the vacuum pump (rotary vane oil pump) is charged with dry ice-acetone or with liquid nitrogen.

The cooling liquid is preheated to 60° C.

At 500 rpm, the CBD is then allowed to drip into the apparatus within approx. 12 h.

At a vacuum of 0.02 to 0.3 mbar, 1760 g of distillate and 178.2 g of distillation residue collect in the particular receivers, as do 12.1 g of condensate in the cold trap.

2. Silica Gel Filtration (Chromatography)

Silica gel or other chromatographic adsorbents, for example aluminium oxide, can retain many impurities which prevent the crystallization of CBD when adsorbed crude CBD is eluted with a suitable solvent.

Suitable solvents are hydrocarbons, halogenated hydrocarbons, esters, ethers and ketones having up to 20 carbon atoms, and mixtures thereof with one another.

For performance, one part by weight of the CBD to be purified is dissolved in a suitable first solvent, such as n-heptane, and this solution is applied to a silica gel bed composed of one part by weight, preferably two parts by weight, of silica gel for chromatography.

The first solvent is allowed to elute and the silica gel bed is then eluted (washed) with a suitable solvent or mixture, for example one part by volume of dichloromethane and 4 parts by volume of heptane, until CBD is no longer detectable in the eluate.

Evaporative concentration of the eluate affords the purified CBD; the retained impurities can be disposed of with the spent silica gel.

Collecting fractions allows qualities of CBD of different purity to be prepared and the impurities to be eluted separately from the CBD.

Crystallization and Recrystallization:

Crude CBD, preferably CBD prepurified by distillation or silica gel filtration, can be crystallized by dissolving in a suitable solvent, cooling the solution and seeding.

This purification process has low losses if conducted appropriately and gives outstanding purifying action.

Suitable solvents for crystallization are hydrocarbons having three to 30 carbon atoms, preferably straight-chain hydrocarbons such as n-pentane, n-hexane, n-heptane.

Additionally suitable are highly fluorinated or partly fluorinated linear hydrocarbons, and esters of saturated or unsaturated linear carboxylic acids having one up to 36 carbon atoms with linear mono-, di- or oligoalcohols, for example glycerol, and mixtures of the solvents mentioned.

Example:

1755.5 g of distilled crude CBD (amorphous) are dissolved while heating and stirring in 7.6 l of n-pentane.

The solution is cooled and repeatedly seeded with constant stirring.

At a temperature of <20° C., the CBD begins to crystallize.

With further stirring, the mixture is cooled to minus 38° C. and the crystal slurry of CBD which forms is filtered with suction under cold conditions and washed with 1.5 l of cold n-pentane.

After drying, 1313.3 g of crystalline CBD remain.

Recrystallization:

1010.1 g of crystalline CBD are analogously dissolved in 3.8 l of warm n-pentane. Cool to minus 38° C. while stirring and seeding.

Filter off with suction and wash with 1.5 l of ice-cold (<minus 30° C.) n-pentane.

Yield after drying 985.7 g.

Step 3: Acid-Catalysed Isomerization (Reaction “c”)

If an acid-catalysed rearrangement (isomerization/epimerization) leads to the desired end product, this can in principle be carried out before or after the decarboxylating hydrolysis (reaction “b”).

In principle, the same acids and solvents as in the condensation step “a” are used.

The selection of the acid, of the solvent and of the appropriate temperature allows the reaction to be controlled in the desired manner.

The abovementioned isomerizations (ring closure reactions, epimerizations and rearrangements on the carbon skeleton) are explained here using the example of the synthesis of dronabinol from cannabidiol.

Example of an Acid-Catalysed Isomerization (Step “c”):

In a 2 l three-neck flask with stirrer, dropping funnel and drying tube, 31 g of cannabidiol are dissolved in 1.0 l of dichloromethane.

Optionally, an alkaline desiccant such as potassium carbonate or basic aluminium oxide can be added.

Then a solution of 5.0 g of boron trifluoride etherate in 100 ml of dichloromethane is added dropwise while stirring.

The mixture is stirred at room temperature and the progress of the reaction is checked with the aid of gas chromatography at intervals of 15 min.

Toward the end of the reaction, the delta-8-tetrahydrocannabinol content rises to a greater than proportional degree.

When the delta-8-tetrahydrocannabinol content is 2% relative to delta-9-tetrahydro-cannabinol, the reaction is stopped by adding 300 ml of 5% sodium hydrogencarbonate solution.

The mixture is stirred for a further hour, the phases are separated, and the organic phase is washed successively with 300 ml of 5% sodium hydrogencarbonate solution and twice with 300 ml each time of deionized water.

Subsequently, the organic phase is concentrated and the residue is purified by chromatography on silica gel.

Yield: 27.9 g (90% of theory) of pure dronabinol.

1. Process for preparing a compound of the general formula Ia, Ib or Ic and diastereoisomers thereof 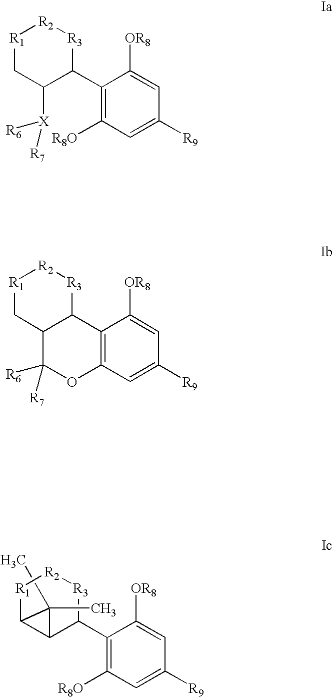
by decarboxylating hydrolysis of the compound of the general formula IIa, IIb, IIc or IId 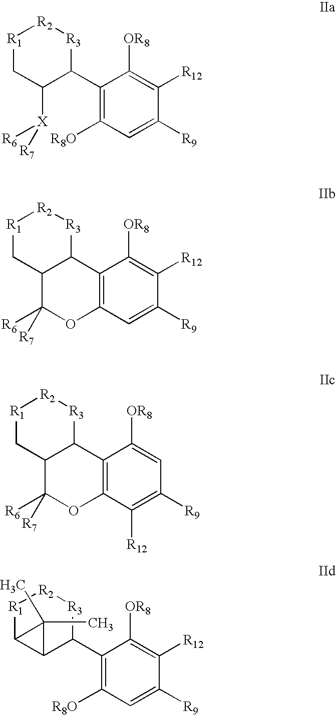
where
R1-R2 or R2-R3 may be a C—C double bond, and
R1 and R3 are each C or CH, and
R2 is either C═O or an R10-C—R11 group,
where
R10 and R11 are each independently H or a lower C₁-C₄ alkyl group when a double bond is not present between R1 and R3, or, if a double bond is present between R1 and R3, one of the R10 and R11 groups is absent and the other is as defined above;
X is either C when R6 is a ═CH₂ group and R7 is a CH₃ group, or
X is a CR4 group where R4 is H or a lower alkyl group, CH or a C—O—R5 group, and R5 is H, a C₁-C₁₆ alkyl group or a protecting group;
R6 and R7 are each a CH₃ group or at least one of the R6 and R7 groups is a CH2=group and the other is a CH₃ group,
R8 is a C₁-C₁₆ alkyl group, H or a protecting group,
R9 is a C1-C16 or O—C₁-C₁₆ group, where C₁-0₁₆ is a straight or branched alkyl chain which has one or more double or triple bonds at any position or may have substituents such as deuterium or halogen atoms, phenyl, substituted phenyl, cycloalkyl, nitrile, alkoxy or a keto group,
R12 is a CO₂—R13 group, and
R13 is H, a C₁-C₁₆ alkyl group or a protecting group.

2. Process according to claim 1, wherein the decarboxylating hydrolysis is performed in an acidic medium.

3. Process according to claim 1, wherein the decarboxylating hydrolysis is performed in an alkaline medium.

4. Process according to claim 3, wherein the hydrolysis is performed under pressure.

5. Process according to claim 4, wherein the process is performed using C1-C5 lower alcohols as solvents with ammonia.

6. Process according to claim 3, wherein the process is performed in aqueous ammonia with phase transfer catalysts or emulsifiers.

7. Process according to claim 3, wherein the process is performed at atmospheric pressure in a solvent which boils above 100° C.

8. Process according to claim 7, wherein at least one of the following solvents is used: DMF, DMSO, sulpholane, furfurol, di- and tetrahydrofuran, 2-methoxytetrahydrofuran, hexamethylenephosphoramide, acetamide, amides having up to 12 carbon atoms, tetramethylurea, ethylene glycol, and the mono- and bisethers thereof, ethanolamine, ethylenediamine, propylene glycol and ethers thereof having up to 20 carbon atoms, glycerol and glyceryl ethers having up to 30 carbon atoms, 1,2-butanediol, 1,3-butanediol, 1,4-butanediol, diethylene glycol and ethers thereof, triethylene glycol and ethers thereof, polyethylene glycol, polyvinylpyrrolidone with or without water.

9. Process according claim 3, wherein one or more of the following bases are used for alkaline hydrolysis:
hydroxides of alkali metals and alkaline earth metals, quaternary ammonium salts, carboxylates having up to 30 carbon atoms, phenoxides, phosphates, phosphites, sulphides, hydrogensulphides, mercaptides having up to 30 carbon atoms, sulphites, hydrogensulphites, cyanides of alkali metals and quaternary ammonium salts having 4 to 48 carbon atoms, ammonia, organic alkylamines, arylamines or aromatic or nonaromatic heterocyclic amines having up to 36 carbon atoms, and the salts of abovementioned amines, borates, tetraborates.

10. Process according to claims 3, wherein the decarboxylation catalysts used are transition metals and salts thereof, preferably stainless steel powder or silver powder.

11. Process according to claim 1, wherein the compound of the formula Ia, Ib or Ic is prepared by an acid-catalysed condensation of a suitable unsaturated terpene precursor with the compound of the general formula III where R8, R9 and R12 are each as defined in claim 1

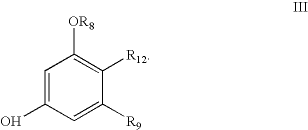

12. Process according to claim 11, wherein the condensation is performed in the presence of acetals of N,N-dimethylformamide, for example N,N-dimethylformamide dineopentyl acetal or other water-releasing reagents, preferably propanephosphonic anhydride, are added or applied to a support material, for example aluminium oxide, or in the presence of at least one of the following Brønsted or Lewis acids: perchloric acid, hydrohalic acids (HF, HCl, HBr, HI), sulphuric acid, hydrogensulphates, phosphoric acid and the acidic salts thereof, pyro- and polyphosphoric acids, organic carboxylic and sulphonic acids having one up to 30 carbon atoms and one or more acidic groups, and acidic groups bonded to polymeric supports, for example acidic ion exchangers and mixtures of the acids mentioned, namely formic acid, oxalic acid, trifluoroacetic acid, p-toluenesulphonic acid, the cations of alkaline earth metals and earth metals, and also transition metals; the halogen compounds and other trivalent compounds of elements of the third main group, such as boron trifluoride and other boron-halogen compounds and complexes thereof, aluminium halides such as anhydrous aluminium chloride; salts and halogen compounds of transition metals such as titanium tetrachloride, zinc chloride, zinc trifluoromethanesulphonate; halogen compounds of elements of the fourth and fifth and sixth main groups, for example tin tetrachloride, phosphorus trichloride, phosphorus pentachloride, phosphorus oxychloride, antimony pentafluoride, thionyl chloride, sulphuryl chloride.

13. Process according to claim 11, wherein the condensation is performed in at least one of the following water-miscible or water-immiscible solvents: hydrocarbons having up to 30 carbon atoms, halogenated hydrocarbons having up to 20 carbon atoms, for example dichloromethane or chloroform, ethers, for example 2-methoxytetrahydrofuran, alcohols, carboxylic acids having up to 16 carbon atoms, amides having up to 20 carbon atoms, esters having up to 60 carbon atoms, carbon dioxide, sulphur dioxide, water, water with a phase transfer catalyst, the acidic catalysts alone.

14. Process according to claims 11, wherein the decarboxylating hydrolysis according to claim 1 is preceded or followed by an acid-catalysed rearrangement.

15. Process according to claim 14, wherein the rearrangement is an epimerisation.

16. Process for preparing a compound of the general formula Ia, Ib or Ic according to claim 1, comprising the following steps:
a) reacting according to claim 11 a compound of the general formula III with a suitable unsaturated terpene in order to obtain a compound of the general formula IIa, IIb, IIc or IId according to claim 1;
b) performing the decarboxylating hydrolysis according to any of claims 1-10; and
c) performing an acid-catalysed rearrangement,
step c) being performable before step b).
